(19) United States
(12) Patent Application Publication
(10) Pub. No.: US 2016/0166757 A1
(43) Pub. Date: Jun. 16, 2016

(54) LIQUID DETECTING DEVICE, ELECTRODE CONNECTOR FOR SAME, LIQUID DETECTING SYSTEM, AND LIQUID DETECTING METHOD

(71) Applicant: TATSUTA ELECTRIC WIRE & CABLE CO., LTD., Higashiosaka-shi, Osaka (JP)

(72) Inventors: Akihiko KOYAMA, Higashiosaka-shi, Osaka (JP); Keisho SHINOHARA, Higashiosaka-shi, Osaka (JP); Junichi INOUE, Higashiosaka-shi, Osaka (JP); Takashi MORITA, Higashiosaka-shi, Osaka (JP)

(21) Appl. No.: 14/436,841

(22) PCT Filed: Oct. 18, 2013
(86) PCT No.: PCT/JP2013/078309

§ 371 Date: Apr. 17, 2015

(30) Foreign Application Priority Data
Oct. 19, 2012 (JP) 2012-231722
Oct. 19, 2012 (JP) 2012-231723
Jan. 30, 2013 (JP) 2013-015225
Mar. 21, 2013 (JP) 2013-058748

(51) Int. Cl. A61M 1/36; G01N 27/08

(52) U.S. Cl. CPC A61M 1/3669; G01N 27/08; A61M 2205/15

(57) ABSTRACT

Liquid leakage is certainly detected and abnormality in an installation state is detected. A liquid detecting device 10 includes a liquid detection sensor 1 including an insulation sheet 14 exhibiting conductivity in the presence of liquid, electrode members 15 electrically isolated from each other, and a resistance member 18 connected to join the electrode members 15 together. In the liquid detecting device 10, the measurement device 2 measures a value of resistance between the electrode members 15. Based on the value of resistance between the electrode members 15, the liquid detecting device 10 identifies a liquid leakage state in which the insulation sheet 14 exhibits conductivity, a disconnected state in which the electrode members 15 are disconnected from the measurement device 2, and a measurement preparation completed state, and outputs identification information which corresponds to each identified state.

### TECHNICAL FIELD

The present invention relates to a liquid detecting device configured to detect liquid such as water and oil, a liquid detecting system, and a liquid detecting method.

### BACKGROUND

A known sensor for detecting liquid leakage is recited in PTL 1. PTL 1 discloses a flexible liquid leakage detector in which two or more electrode foils which are separated from one another and disposed side by side are sandwiched between a synthetic resin tape and a synthetic resin non-woven tape so as to be fixed to one another, and an adhesive material layer having an arbitrary shape is provided on a surface of the synthetic resin non-woven tape which surface is in contact with skin.

In such a liquid leakage detector, the resistance between electrode members (electrode foils) is infinite in a normal measurement preparation completed state in which no liquid leakage occurs, whereas the resistance is low in a liquid leakage state because the electrode members are electrically connected with each other by a wet insulation sheet (non-woven fabric made of synthetic resin). Accordingly, a detector for detecting the resistance between the electrode members (electrode foils) is connected, and the liquid leakage state is detected based on a variation in the resistance.

### CITATION LIST

### Patent Literature

[PTL 1] Japanese Utility Model Publication No. 79468/1993 (Jitsukaihei 5-79468)

### SUMMARY OF THE INVENTION

### Technical Problems

In the known arrangement above, the resistance between the electrode members always becomes infinite when, for example, the connection between the detector and the electrode members is mechanically cut off or the detector drops off from the electrode members. That is to say, in such a case, even if the liquid leakage state occurs, the liquid leakage detector cannot detect the liquid leakage state and keeps indicating the measurement preparation completed state.

The present invention has been done to solve the problem above, and an object of the present invention is to provide a liquid detecting device, a liquid detecting system, and a liquid detecting method, by which liquid leakage is certainly detected and disconnection regarding an installation state is detectable.

### Technical Solution

A liquid detecting device of the present invention includes: a liquid detection sensor including an insulation sheet which exhibits conductivity in the presence of liquid, a plurality of electrode members which are provided on one surface of the insulation sheet in a contacting manner and are electrically isolated from each other, and a resistance member connected to join the electrode members together; a resistance value detection part which is detachably connected to the electrode members via a signal line and is configured to detect a value of resistance between the electrode members; a state identification part which identifies, based on the value of resistance between the electrode members, a liquid leakage state in which the insulation sheet exhibits conductivity, a disconnected state in which the electrode members are disconnected from the resistance value detection part, and a measurement preparation completed state; and an information output part which is configured to output identification information which corresponds to each state identified by the state identification part.

According to the configuration above, the liquid detection sensor is arranged such that the electrode members are connected with each other by the resistance member on one surface of the insulation sheet. On this account, when the insulation sheet does not exhibit conductivity, the electrode members are electrically connected with each other only by the resistance member. In the meanwhile, when the insulation sheet exhibits conductivity, the electrode members are electrically connected with each other by the insulation sheet and the resistance member. With this configuration, the resistance value detection part detects a different value of resistance between the electrode members depending on whether the insulation sheet exhibits conductivity. When the resistance value detection part is disconnected from the electrode members, the resistance value detection part is disconnected from a connection circuit of electrode members composed of the electrode members and the resistance member on the insulation sheet, and therefore detects a value of resistance in the disconnected state. As a result, based on the value of resistance detected by the resistance value detection part, the state identification part identifies the liquid leakage state in which the insulation sheet exhibits conductivity, the disconnected state in which the electrode members are disconnected from the resistance value detection part, and the measurement preparation completed state. On this account, an operation to connect the liquid detection sensor with the resistance value detection part is accurately performed based on the identification information output from the information output part, and states of the liquid detection sensor can be monitored from the outside.

In the above-described liquid detection sensor of the liquid detecting device of the present invention, the resistance member may have a value of resistance higher than a value of resistance when the insulation sheet exhibits conductivity and lower than a value of resistance when the electrode members are disconnected from the resistance value detection part.

According to the configuration above, the resistance value detection part detects values of resistance which have a relationship as follows: the value of resistance in the liquid leakage state<the value of resistance in the measurement preparation completed state<the value of resistance in the disconnected state. Based on these values of resistance, the state identification part is able to identify the liquid leakage state in which the insulation sheet exhibits conductivity, the disconnected state in which the electrode members are disconnected from the resistance value detection part, and the measurement preparation completed state.

In addition to the above, in the liquid detecting device of the present invention, the resistance value detection part may include a sandwiching part which is able to sandwich the liquid detection sensor and a connector-side electrode member which is provided at the sandwiching part, in contact with the electrode members when the liquid detection sensor is sandwiched, and is electrically connected with the signal line.

According to this configuration, as the sandwiching part of the electrode connector sandwiches the liquid detection sensor, the connector-side electrode member provided at the sandwiching part is brought in contact with the electrode members. With this, the signal line of the resistance value detection part is electrically connected with the electrode members via the connector-side electrode member. This makes it possible to easily connect the resistance value detection part to the liquid detection sensor in a detachable manner. Furthermore, when a predetermined force (pulling the electrode connectors or the liquid detection sensor) is applied from the outside, an electric value of resistance becomes infinite at the time of the disconnection of the liquid detection sensor from the sandwiching part, and hence this disconnection is detectable.

In addition to the above, in the liquid detecting device of the present invention, the liquid detection sensor may be installed to a puncture position which is punctured by a puncture appliance, and the electrode connector may include a grip part which is attachable to the puncture appliance.

According to the configuration above, when a force of pulling the puncture appliance at puncture position is applied and the puncture appliance becomes in an abnormal installation state, for example, the puncture appliance falls off, the liquid detecting device detects the disconnected state because the connection between the electrode connector attached to the puncture appliance by the grip part and the electrode members is canceled. As a result, in the case that the abnormal installation state of the puncture appliance occurs, the liquid detecting device is able to at least detect the abnormality based on the detection of the disconnected state.

In addition to the above, in the liquid detecting device of the present invention, the information output part may include an output unit which is configured to output the identification information by sound and/or light.

According to the configuration above, because the identification information is output by sound, light, or both sound and light, the state of the liquid detection sensor is easily determined.

In addition to the above, in the liquid detecting device of the present invention, the information output part may include a terminal-side communication unit which is configured to transmit at least the identification information.

According to this configuration, because the identification information is transmitted from the terminal-side communication unit, the state of the liquid detecting device can be monitored based on the transmitted identification information, even from a location remote from the liquid detecting device. Furthermore, for example, when the identification information is transmitted to a dialyzer, the dialyzer receives the identification information which indicates the disconnected state or the liquid leakage state. This makes it possible to quickly restrain liquid leakage by stopping the pump of the dialyzer by which blood is circulated.

In addition to the above, in the liquid detection sensor of the present invention, the terminal-side communication unit may transmit unique ID information together with the identification information.

According to this configuration, because it is possible to specify which liquid detection sensor is the source of data transmission based on the ID information, remotely monitoring plural liquid detecting devices is achieved when the ID information is preliminarily associated with each installation location.

In addition to the above, a liquid detecting system of the present invention includes: the liquid detecting device; a monitoring device which is configured to monitor the liquid detecting device, the monitoring device including: a monitoring communication part which is connected to the terminal-side communication unit of the liquid detecting device to perform data communication with the terminal-side communication part; a monitoring storage part which is configured to store the ID information of the liquid detecting device in association with installation location information; a display which is configured to display the identification information and the installation location information; and a display processor which is configured to cause the display to demonstrate identification information which is input through the monitoring communication part and the installation location information which corresponds to the ID information input together with the identification information.

According to this configuration, it is possible to monitor the liquid detecting device from a remote location.

In addition to the above, a liquid detecting method of the present invention includes: a step of setting a liquid detection sensor including an insulation sheet which exhibits conductivity in the presence of liquid, a plurality of electrode members which are provided on one surface of the insulation sheet in a contacting manner and are electrically isolated from each other, and a resistance member connected to join the electrode members together to a liquid leakage detection target; a resistance value detection step of establishing detachable connection with the electrode member via a signal line and detecting a value of resistance between the electrode members; a state identification step of, based on the value of resistance between the electrode members, identifying a liquid leakage state in which the insulation sheet exhibits conductivity, a disconnected state in which the electrode members are disconnected from the resistance value detection part, and a measurement preparation completed state; and an information output step of outputting identification information which corresponds to each state identified by the state identification part.

According to the configuration above, the liquid detection sensor is configured such that the electrode members are connected with each other by the resistance member on one surface of the insulation sheet. On this account, when the insulation sheet does not exhibit conductivity, the electrode members are electrically connected with each other only by the resistance member. In the meanwhile, when the insulation sheet exhibits conductivity, the electrode members are electrically connected with each other by the insulation sheet and the resistance member. Therefore, it is possible in the resistance value detection step to detect a different value of resistance between the electrode members depending on whether the insulation sheet exhibits conductivity. When the resistance value detection part is disconnected from the electrode members, the resistance value detection part is disconnected from a connection circuit composed of the electrode members and the resistance member on the insulation sheet, and therefore a value of resistance in the disconnected state, which is higher than the value of resistance of the connection circuit, is detected in the resistance value detection step. As a result, based on the value of resistance detected in the resistance value detection step, the liquid leakage state in which the insulation sheet exhibits conductivity, the disconnected state in which the electrode members are disconnected from the resistance value detection part, and the measurement preparation completed state are identified in the state identification step. On this account, an operation to connect the liquid detection sensor with the resistance value detection part is accurately performed based on the identification information output in the information output step, and states of the liquid detection sensor can be monitored from the outside.

### Advantageous Effect of Invention

Liquid leakage can be certainly detected as well as completion of measurement preparation and connection release.

### BRIEF DESCRIPTION OF THE DRAWINGS

FIG. 1 shows the outline of a liquid detecting device.

FIG. 2 shows the outline of a liquid detecting system.

FIG. 3 shows the cross-sectional structure of a liquid detection sensor.

FIG. 4 is a perspective diagram of an electrode connector.

FIG. 5 is a block diagram showing the configuration of a measuring device.

FIG. 6 shows an installation information table.

FIG. 7 is a flowchart of a liquid leakage detection program run by the measuring device.

### PREFERRED EMBODIMENT OF INVENTION

A preferred embodiment of the present invention will be described with reference to figures.

### (Outline of Liquid Detecting Device)

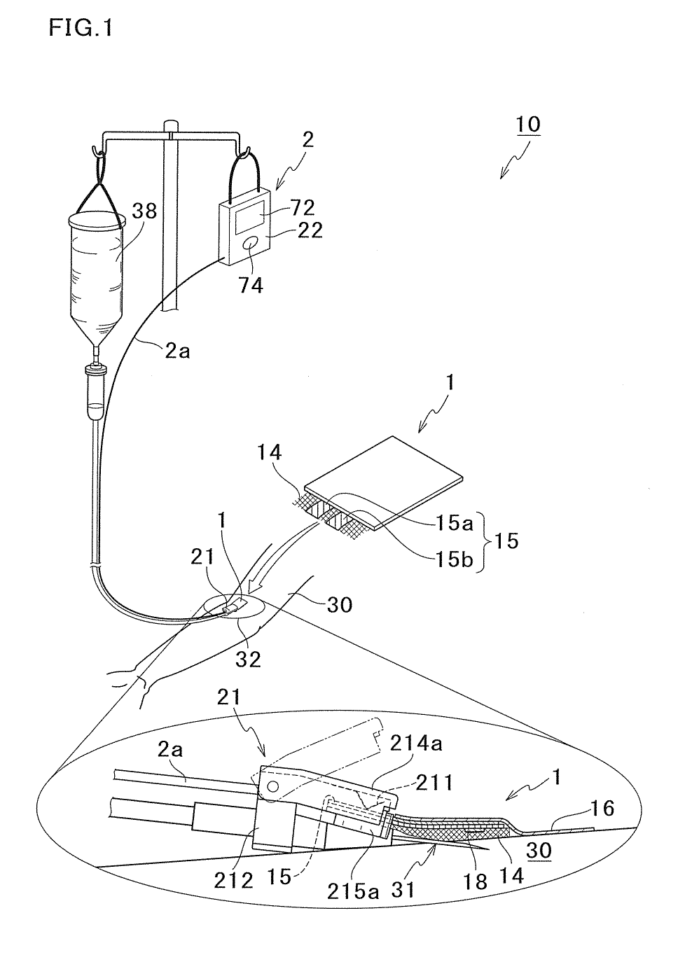

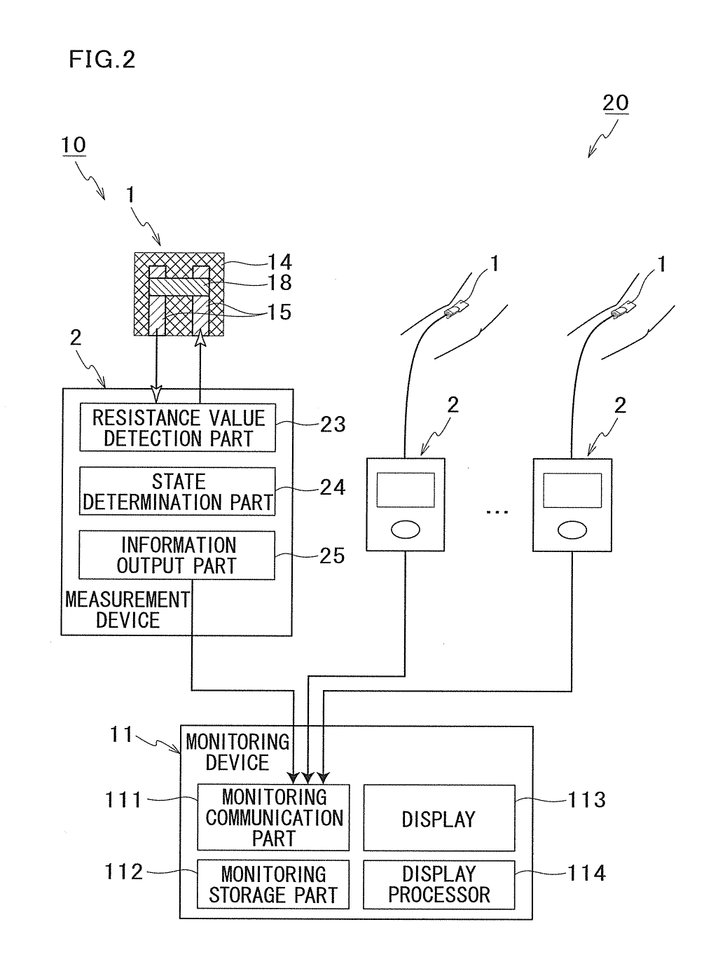

As shown in FIG. 1 and FIG. 2, a liquid detecting device 10 of the present embodiment includes a liquid detection sensor 1 and a measurement device 2. In the liquid detecting device 10, the measurement device 2 measures a value of resistance between plural (two in the present embodiment) electrode members 15 provided in the liquid detection sensor 1. The liquid detecting device 10 identifies the state of the liquid detection sensor 1 with reference to the value of resistance between the electrode members 15.

To be more specific, the liquid detection sensor 1 includes an insulation sheet 14 which exhibits conductivity in the presence of liquid, the electrode members 15 (electrode members 15a and 15b) which are provided on one surface of the insulation sheet 14 in a contacting manner and are electrically isolated from each other, and a resistance member 18 connected to join the electrode members 15 together. Furthermore, the measurement device 2 includes a resistance value detection part 23 which is detachably connected to the electrode members 15 via a signal line to detect a value of resistance between the electrode members 15, a state identification part 24 which is configured to identify the state of the liquid detection sensor 1 based on the value of resistance between the electrode members 15, and an information output part 25 which is configured to output identification information corresponding to each state identified by the state identification part 24.

The “liquid” refers to a liquid-state detection target to be detected by the liquid detection sensor 1, and the material and the physical properties thereof are not particularly limited on condition that it is in a liquid state. The liquid state indicates fluidity with which the insulation sheet 14 can be impregnated with liquid and retain liquid. Examples of the “liquid” include a body fluid, a liquid medicine, pure water, water including impurities, an acid, an alkali, oil, and an organic matter such as an organic solvent. The physical properties of the “liquid” can be liquefied substance under the environmental temperature that the liquid detection sensor 1 can function.

The states of the liquid detection sensor 1 identified by the liquid detecting device 10 include at least the following three states: a “liquid leakage state”, a “disconnected state”, and a “measurement preparation completed state”.

The liquid leakage state is a state in which the insulation sheet 14 exhibits conductivity. That is, the liquid leakage state is a state in which the insulation sheet 14 exhibits conductivity on account of the presence of liquid in the insulation sheet 14 and the electrode members 15 are electrically connected with each other by the insulation sheet 14. For this reason, the value of resistance between the electrode members 15 detected by the resistance value detection part 23 is a value of resistance of the insulation sheet 14, which is lower than a value of resistance of the resistance member 18.

The disconnected state is a state in which the electrode members 15 are disconnected from the resistance value detection part 23. That is to say, in the disconnected state, because the resistance value detection part 23 is physically detached from the electrode members 15, the value of resistance between the electrode members 15 cannot be measured, and hence the value of resistance detected by the resistance value detection part 23 is infinite in theory.

The measurement preparation completed state is a state which is neither the liquid leakage state nor the disconnected state. In other words, the electrode members 15 are not electrically connected with each other by the insulation sheet 14, and the electrode members 15 are connected with the electrode connector 21. Because in the measurement preparation completed state the electrode members 15 are connected with each other only by the resistance member 18, the relationship between the values of resistance in the respective states detected by the resistance value detection part 23 is as follows: a value of resistance in the liquid leakage state<a value of resistance in the measurement preparation completed state<a value of resistance in the disconnected state.

As such, in the liquid detection sensor 1, the electrode members 15 are connected with each other by the resistance member 18 on one surface of the insulation sheet 14. Therefore, when the insulation sheet 14 does not exhibit conductivity, the electrical connection between the electrode members 15 is achieved only by the resistance member 18. In the meanwhile, when the insulation sheet 14 exhibits conductivity, the electrical connection between the electrode members 15 is achieved by the insulation sheet 14 and the resistance member 18. In this way, the value of resistance between the electrode members 15 detected by the resistance value detection part 23 varies depending on whether the insulation sheet 14 exhibits conductivity. When the resistance value detection part 23 is disconnected from the electrode members 15, the resistance value detection part 23 detects the value of resistance in the disconnected state because the resistance value detection part 23 is detached from a connection circuit of electrode members 15 composed of the insulation sheet 14 and the resistance member 18. As a result, based on the value of resistance detected by the resistance value detection part 23, the state identification part 24 identifies the liquid leakage state in which the insulation sheet 14 exhibits conductivity, the disconnected state in which the electrode member 15 is disconnected from the resistance value detection part 23, and the measurement preparation completed state which is different from the former two states. This makes it possible to accurately connect the liquid detection sensor 1 with the resistance value detection part 23 based on the identification information output from the information output part 25, and to monitor the states of the liquid detection sensor 1 from the outside.

In addition to the above, as shown in FIG. 2, a liquid detecting system 20 including plural liquid detecting devices 10 and a monitoring device 11 which is able to perform data communication with each of the liquid detecting devices 10 is configured in the present embodiment. In the present embodiment, the liquid detecting devices 10 are connected with the monitoring device 11 by wireless communication. The number of the liquid detecting devices 10 in the liquid detecting system 20 may not be plural, and at least one liquid detecting device 10 may be provided to be connectable. The data communications between the liquid detecting devices 10 and the monitoring device 11 are not limited to wireless communication, and may be established by wire. The standard of the data communications is not particularly limited.

### (Structure of Liquid Detection Sensor)

As shown in FIG. 1, the liquid detection sensor 1 includes the insulation sheet 14, the paired electrode members 15 (electrode members 15a and 15b), and the resistance member 18. In the present embodiment, a puncture position 30 (of a human arm, leg, etc.) punctured by a puncture appliance 31 (such as an indwelling needle and a winged needle) is illustrated as an example of the installation target of the liquid detection sensor 1. In regard to this example, the puncture appliance 31 may come out during treatment such as dialysis, blood transfusion, and drip infusion so that blood or liquid medicine may leak out from the puncture appliance 31 or the puncture position 30. In such a case, because the liquid detection sensor 1 is pasted onto the puncture position 30, it is possible to detect liquid leakage of blood or liquid medicine and improper installation of the liquid detection sensor 1. In this regard, because the liquid detection sensor 1 is directly pasted onto the puncture position, liquid leakage is detectable even if a small amount of liquid leakage occurs.

The liquid detection sensor 1 is preferably sterilized for medical use. In particular, the liquid detection sensor 1 is preferably sterilized by ethylene oxide gas (EGG).

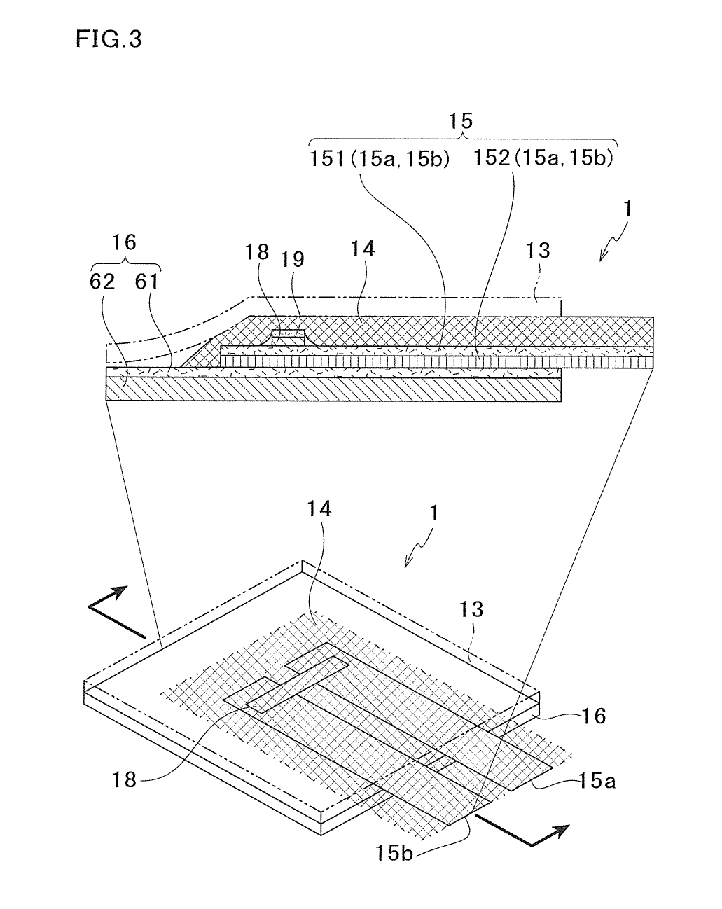

To be more specific, as shown in FIG. 3, the liquid detection sensor 1 is configured by laminating the insulation sheet 14, an adhesive layer 19, the resistance member 18, the two electrode members 15a and 15b, and an adhesive member 16. On condition that the electrode members are connected with each other by the insulation sheet and the resistance member, the configuration of these members and the order of the layers are not limited to the above.

### (Liquid Detection Sensor: Insulation Sheet)

The insulation sheet 14 exhibits conductivity in the presence of liquid. In other words, the insulation sheet 14 is insulative when, for example, it is not impregnated with liquid, and is conductive in the presence of liquid. For this reason, when the insulation sheet 14 is not impregnated with liquid, the electrode members 15 are not electrically connected with each other by the insulation sheet 14. On the other hand, the electrode members 15 are electrically connected by the insulation sheet 14 when liquid is present in the insulation sheet 14.

The outer shape of the insulation sheet 14 is similar to the outer shape of the liquid detection sensor 1, and is rectangular in plan view. The insulation sheet 14 is smaller in size than the liquid detection sensor 1 and is configured at a central part of the liquid detection sensor 1. The shape of the liquid detection sensor 1 may not be rectangular in plan view. The shape of the liquid detection sensor 1 may be polygonal, e.g., triangular or pentagonal, or may be elliptical or circular. The shape of the insulation sheet 14 may be similar to or different from such a shape of the liquid detection sensor 1.

The insulation sheet 14 has a structure of exhibiting conductivity in the presence of liquid, as well as absorbing and retaining liquid. In other words, the insulation sheet 14 is configured to switch from being insulative to conductive overall, as a result of the permeation of liquid.

The “liquid absorbing and retaining structure” of the insulation sheet 14 is not limited to any material and shape if the structure can be impregnated with the liquid, a detection target. Examples of the liquid absorbing and retaining structure include a non-woven fabric structure, a porous structure having open cells or the like, a structure in which one or more hole is formed in a non-porous material, and a structure in which one or more slit is formed in a non-porous material. When the insulation sheet 14 is made of non-woven fabric or paper, high accuracy of the liquid detection sensor 1 can be achieved because the state of insulation sheet 14 can be impregnated and change from being insulative to conductive even with a small amount of the liquid due to capillary phenomenon.

The material of the insulation sheet 14 is not particularly limited if the material has high electric resistance when not in contact with liquid. For example, the insulation sheet 14 may be made of non-woven fabric, a gauze, a bandage, an adhesive plaster, or a paper tape.

To be more specific, examples of the material of the insulation sheet 14 include vegetable fibers (cellulose fibers) such as cloth (cotton, hemp, etc.) and paper, synthetic fibers (such as rayon and cupra), ceramics, engineering plastics, and porous materials (such as sponge). Examples of the engineering plastics include polypropylene, cross-linked polyethylene, polyester, polybenzimidazole, aramid, polyimide, polyimidoamide, polyetherimide, polyphenylene sulfide (PPS), polyethylene naphthalate (PEN), and polyethylene terephthalate (PET).

To be further specific, for the insulation sheet 14, non-woven fabric made of polyester resin produced by Unitika Limited. (®: MARIX) may be used. This non-woven fabric is hydrophilic because the resin adhering the polyester fibers is water-soluble acrylic resin. The non-woven fabric above is produced by spun bonding. In the non-woven fabric with the item number #20507WTD, the total weight is 50 g/m² and the average thickness is 155 μm. In the non-woven fabric with the item number #20604FLD, the total weight is 60 g/m² and the average thickness is 150 μm. In the non-woven fabric with the item number #10606WTD, the total weight is 60 g/m² and the average thickness is 215 μm (with bulkiness).

The thickness of the insulation sheet 14 is preferably 10 to 3000 μm. The insulation sheet 14 preferably has lyophilicity to liquid which is a detection target. For example, when the detection target liquid is water, the insulation sheet 14 is preferably hydrophilic. With the lyophilicity, even a small amount of liquid permeates the insulation sheet 14 and changes the insulation sheet 14 from being insulative to conductive. For this reason, the liquid is detectable even if the amount thereof is small, and the time required to complete the detection is shortened.

In the insulation sheet 14, the material itself of the insulation sheet 14 may be lyophilic, or a lyophilic layer may be formed on the surface of a lyophobic material. For example, the insulation sheet 14 may be arranged such that, a surfactant which is surface-active against the liquid may be adhered to at least a portion of a contact part where the liquid absorbing and retaining structure is in contact with the liquid. In such a case, the liquid detection sensor 1 is able to detect different types of detection targets such as water and oil, by selecting a type of surfactant corresponding to each type of the liquid to be detected.

In addition to the above, the insulation sheet 14 may include a colored member which changes its color in the presence of liquid. The colored member is, for example, arranged such that a colorant such as dye is sealed in a capsule composed of a solvent such as water and oil, etc. so as to be soluble in liquid. In this case, as the sealed colorant leaks out when the capsule is dissolved by the liquid, the color of the insulation sheet 14 changes. In this way, the liquid detection sensor 1 configured in this manner allows visual detection of liquid leakage.

In addition to the above, to the insulation sheet 14, a soluble material (inorganic salts such as sodium chloride, sodium sulfate, calcium chloride, and magnesium hydroxide) which dissolves in liquid and ionized may be adhered. In such a case, even if the liquid itself is not conductive (e.g., pure water and oil), the soluble material ionized by the liquid changes the insulation sheet 14 to be conductive.

### (Liquid Detection Sensor: Electrode Member)

The electrode members 15a and 15b are provided to be in contact with one surface of the insulation sheet 14. The electrode members 15a and 15b are configured to be parallel in longitudinal direction. The contact state between the electrode members 15 and the insulation sheet 14 may be achieved by adhesion or abutting them with each other. The electrode members 15a and 15b are provided at a predetermined interval. Accordingly, the electrode members 15 are electrically separated from each other. The predetermined interval refers to an interval with which malfunction does not occur owing to the reaction to the moisture in the atmosphere around the liquid detection sensor 1. For this reason, instead of parallel configuration, the electrode members 15 may be comb-shaped or fence-shaped.

The electrode members 15a and 15b is formed by laminating a metal layer 152 and a conductive adhesive layer 151. the electrode members 15a and 15b is therefore adhesive to one surface side of the insulation sheet 14 so that it is adhered to one surface of the insulation sheet 14 in a contact manner by its adhesiveness.

The metal layer 152 may be made of any material on condition that the metal layer 152 has conductivity. As the metal material forming the metal layer 152 may contain any one of the nickel, copper, silver, tin, gold, palladium, aluminum, chromium, titanium, zinc, or an alloy containing two or more of such materials. The material is preferably metal such as aluminum and copper.

The conductive adhesive layer 151 includes resin and conductive particles. Examples of the resin include acrylic resin, silicon resin, thermoplastic elastomer resin, rubber resin, and polyester resin. Specific examples of the resin are KP-1581, KP-1104, KP-2074, and SZ-6153 produced by NIPPON CARBIDE INDUSTRIES CO., INC., and AR-2172-M3 produced by VIGteQnos Co., Ltd. Each conductive particle is partially or entirely formed by the metal material.

Examples of the material of the conductive particles include copper powder, silver powder, nickel powder, silver-coated copper powder (Ag-coated Cu powder), gold-coated copper powder, silver-coated nickel powder (Ag-coated Ni powder), and gold-coated nickel powder. These types of metal powders can be produced by, for example, water atomization or the carbonyl process. In addition to the above, particles formed by coating metal powder with resin or particles formed by coating resin with metal powder may be used. The conductive particles are preferably the Ag-coated Cu powder or the Ag-coated Ni powder. This is because conductive particles with improved conductivity are obtained with low-cost material.

The electrode members 15 may be formed by printing. For example, the electrode members 15 are easily formed by printing silver ink or the like onto the insulation sheet 14.

In the present embodiment, with the electrode members 15a and 15b, a cord 2a of the measurement device 2 is connected. This allows the measurement device 2 to measure a value of resistance between the electrode members 15a and 15b. Though it does not illustrated in figures, the cord 2a has a pair of signal lines which are electrically isolated from each other, and these signal lines are connected with the electrode members 15a and 15b, respectively.

### (Liquid Detection Sensor: Resistance Member)

The resistance member 18 is provided to connect the electrode members 15 with each other. To be more specific, the resistance member 18 is mounted over the two electrode members 15a and 15b to be in contact with these members, and the resistance member 18 is adhered to the insulation sheet 14 on account of the adhesiveness of the adhesive layer 19 laminated on the resistance member 18. The resistance member 18 is adhered to the insulation sheet 14 by the adhesive layer 19 so as to be sandwiched between the insulation sheet 14 and the electrode members 15a and 15b. The mounting position and state of the resistance member 18 are not particularly limited as long as the electrode members 15 are electrically connected with each other by the resistance member 18.

In the liquid leakage state, the resistance member 18 is configured to have a value of resistance higher than the value of resistance of the insulation sheet 14 when the insulation sheet 14 exhibits conductivity. In the meanwhile, in the measurement preparation completed state, the resistance member 18 is configured to have a value of resistance lower than the value of resistance when the electrode members 15 are disconnected from the resistance value detection part 23. Furthermore, in the disconnected state, a value of resistance measured by the resistance value detection part 23 is higher than the value of resistance in the measurement preparation completed state.

As such, the relationship between the values of resistance detected by the resistance value detection part 23 are as follows: the value of resistance in the liquid leakage state<the value of resistance in the measurement preparation completed state<the value of resistance in the disconnected state. On this account, based on these values of resistance, the state identification part 24 is able to identify the liquid leakage state in which the insulation sheet 14 exhibits conductivity, the disconnected state in which the electrode members are disconnected from the resistance value detection part, and the measurement preparation completed state which is different from these states.

The resistance member 18 may be made of any material as long as it has conductivity and can be configured to have the value of resistance thereof higher than the value of resistance when the insulation sheet 14 exhibits conductivity. Preferably, the resistance member 18 is made of carbon. In particular, carbon ink including carbon black such as Ketjenblack (registered trademark) may be directly printed onto the insulation sheet 14, or the resistance member 18 may be formed by performing printing onto a base substrate and then adhering the base material by adhesive. Examples of the material of the base include vegetable fibers (cellulose fibers) such as fabric (cotton, hemp, etc.) and paper, synthetic fibers (such as rayon and cupra), ceramics, and engineering plastics. Examples of the engineering plastics include polypropylene, cross-linked polyethylene, polyester, polybenzimidazole, aramid, polyimide, polyimidoamide, polyetherimide, polyphenylene sulfide (PPS), polyethylene naphthalate (PEN), and polyethylene terephthalate (PET).

Alternatively, metal particles made of nickel, aluminum, etc. may be adhered to the insulation sheet 14 and the electrode members 15 may be brought in contact with a part to which the metal particles are adhered.

In addition to the above, the resistance member 18 may be a thin film layer including resin and conductive particles. In this case, the resistance member is easily formed by merely applying a conductive adhesive including resin and conductive particles onto the insulation sheet 14 or a base so that a thin film is formed on the insulation sheet 14 or the base. Examples of the resin include acrylic resin, silicon resin, thermoplastic elastomer resin, rubber resin, and polyester resin. Specific examples of the resin are KP-1581, KP-1104, KP-2074, and SZ-6153 produced by NIPPON CARBIDE INDUSTRIES CO., INC., and AR-2172-M3 produced by VIGteQnos Co., Ltd. Each conductive particle is partially or entirely formed by the metal material. Examples of the material of the conductive particles include copper powder, silver powder, nickel powder, silver-coated copper powder (Ag-coated Cu powder), gold-coated copper powder, silver-coated nickel powder (Ag-coated Ni powder), and gold-coated nickel powder. These types of metal powders can be produced by, for example, water atomization or the carbonyl process. In addition to the above, particles formed by coating metal powder with resin or particles formed by coating resin with metal powder may be used. The conductive particles are preferably the Ag-coated Cu powder or the Ag-coated Ni powder. This is because conductive particles with improved conductivity are obtained with low-cost material.

The lower limit of the value of resistance of the resistance member 18 must be suitably configured in accordance with a value of resistance of liquid which is a detection target, and to have a higher value of resistance than that of the insulation sheet 14 when the insulation sheet 14 exhibits conductivity and liquid leakage is detected.

### (Liquid Detection Sensor: Adhesive Member)

The adhesive member 16 is formed by laminating an adhesive 61 and an adhesive film 62. The adhesive member 16 is formed to retain the insulation sheet 14 and the electrode members 15a and 15b and to cover the insulation sheet 14, the electrode members 15a and 15b, and the resistance member 18. Note that, the insulation sheet 14 and the electrode members 15a and 15b protrude for a predetermined length from a longitudinal edge of the adhesive member 16 on which the electrode members 15a and 15b are parallelly mounted. The protruding part is therefore not covered with the adhesive member 16. The adhesive member 16 is adhesive at an exposed part thereof. For this reason, the insulation sheet 14, the electrode members 15a and 15b, and the resistance member 18 of the liquid detection sensor 1 can be easily attached to a desired location through the adhesive member 16. The adhesive film 62 functions as a base film of the adhesive 61. The adhesive film. 62 is provided on the opposite side of the surface where the insulation sheet of the liquid detection sensor 1 paste. The adhesive film 62 is sized to be larger than the insulation sheet 14 and the electrode members 15a and 15b except the protruding part thereof. With this configuration, the adhesive film 62 not only retains the adhesive 61 but also covers the insulation sheet 14 and the electrode members 15a and 15b in the liquid detection sensor 1 under the installation state, so as to protect the insulation sheet 14 and the electrode members 15a and 15b from an external force caused by an impact or a scrape.

In the liquid detection sensor 1, a peelable sheet 13 which has the same outer shape as the adhesive member 16 may be provided. This peelable sheet 13 makes it possible to maintain the adhesiveness of the adhesive 61 for a longtime and to allow the adhesive 61 to exhibit adhesiveness against an installation target of the liquid detection sensor 1 only when necessary. As such, the peelable sheet 13 and the adhesive member 16 protect the insulation sheet 14 and the electrode members 15a and 15b before the liquid detection sensor 1 is installed.

### (Structure of Measurement Device)

The measurement device 2 includes a main body 22 which houses members such as a circuit for detecting a value of resistance and the electrode connector 21 which is connected with the main body 22 by the cord 2a. With this structure, the measurement device 2 realizes a function as the resistance value detection part 23 which is detachably attached to the electrode members 15a and 15b by the cord 2a and detects a value of resistance between the electrode members 15, a function as the state identification part 24 which identifies the state of the liquid detection sensor 1 based on the value of resistance between the electrode members 15, and a function as the information output part 25 which outputs identification information corresponding to each state determined by the state identification part 24. As shown in FIG. 1, the main body 22 is provided with a display 72 and a speaker 74 as the information output part 25. The display 72 is an output unit which is configured to output identification information by means of light, and is constituted by an LED or the like. The speaker is an output unit which is configured to output identification information by means of sound.

The identification information is information based on which state of the liquid detection sensor 1 (the liquid leakage state, the disconnected state, and the measurement preparation completed state) can be identified. Because the liquid leakage state and the disconnected state can be identified by visually checking the liquid detection sensor 1, the identification information may be sufficient if the liquid detection sensor can at least determine whether it is the measurement preparation completed state or not. Because the identification information is output in sound, light, or sound and light, in this way, it is possible to easily identify the state of the liquid detection sensor 1.

In the present embodiment, the display 72 indicates the identification information by turning on, turning off, or flickering the LED. Alternatively, for example, the display may be a liquid crystal display and outputs the identification information as texts, marks, and pictures, etc. The speaker 74 indicates the identification information by notification sound such as buzzer sound. The speaker 74 is not limited to the above. For example, the device that can specifically output the content indicating the identification information by sound.

### (Measurement Device: Electrode Connector)

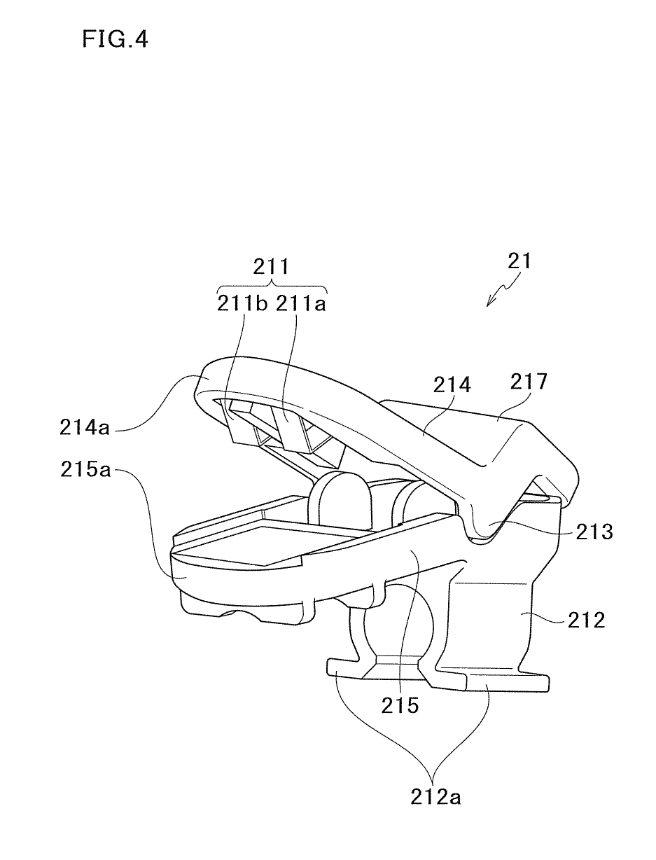

As shown in FIG. 1 and FIG. 4, the electrode connector 21 includes sandwiching parts 214a and 215a which are able to sandwich the liquid detection sensor 1 and connector-side electrode members 211 (connector-side electrode members 211a and 211b) which are provided at the sandwiching part 214a, in contact with the electrode members 15a and 15b when the liquid detection sensor 1 is sandwiched, and electrically connected with the cord 2a. As described above, the cord 2a includes a pair of signal lines. These paired signal lines are connected with the connector-side electrode members 211a and 211b, respectively. This makes it possible to measure a value of resistance between the electrode members 15a and 15b by applying a voltage between the electrode members 15a and 15b and measuring current.

To be more specific, the electrode connector 21 includes paired holders 214 and 215 which are openable/closable by fulcrum 213, a sandwiching part 214a formed in the holder 214, a sandwiching part 215a formed in the holder 215, connector-side electrode members 211 (connector-side electrode members 211a and 211b) provided in the sandwiching part 214a, and a grip part 212 provided in the holder 215. The connector-side electrode members 211 of the electrode connector 21 are electrically connected with the measurement device 2 by the cord 2a.

As the sandwiching part 214a and the sandwiching part 215a sandwich the electrode members 15a and 15b of the liquid detection sensor 1, the connector-side electrode member 211a is in contact with the electrode member 15a whereas the connector-side electrode member 211b is in contact with the electrode member 15b. In this way, each of the electrode members 15a and 15b is electrically connected with the measurement device 2 in an independent manner. Furthermore, the electrode connector 21 can be fixed to the puncture appliance 31 at the grip part 212.

The holders 214 and 215 are positioned to face each other, and the holder 214 is connected with the holder 215 to be rotatable about the fulcrum 213. The holder 214 is biased toward the holder 215 by an unillustrated energizing mechanism such as a spring.

On the tip sides of the holders 214 and 215, the sandwiching parts 214a and 215a for sandwiching the liquid detection sensor 1 are provided. Each of the sandwiching parts 214a and 215a may have a concave-convex teeth part which is engaged with the opposing teeth part. In this case, because the teeth parts bite the liquid detection sensor 1 when the liquid detection sensor 1 is sandwiched between the sandwiching parts 214a and 215a, the liquid detection sensor 1 is firmly fixed.

Each of the connector-side electrode members 211a and 211b is formed to be triangular in shape and protrude toward the holder 215. This facilitates and ensures the contact with the electrode members 15a and 15b.

The connector-side electrode members 211a and 211b may be made of any material as long as these members exhibit conductivity. In other words, the metal material of connector-side electrode members 211a and 211b may be any one of the nickel, copper, silver, tin, gold, palladium, aluminum, chromium, titanium, zinc, or an alloy containing two or more of such materials. The material is preferably metal such as aluminum and copper.

In addition to the above, as shown in FIG. 4, the holder 215 is provided with the grip part 212. The grip part 212 is Q-shaped in cross section view. On this account, when the liquid detection sensor 1 is installed to the puncture position 30 which is punctured by the puncture appliance 31, it is possible to fix the electrode connector 21 to the puncture appliance 31 by attaching the grip part 212 to the puncture appliance 31.

The grip part 212 is made of an elastic material which is elastically deformable, in order to facilitate the fitting of the grip part 212 onto the puncture appliance 31. Examples of the elastic material include resin and polymer which is mainly made of vulcanized rubber. Examples of the resin include polyurethane resin, epoxy resin, polypropylene resin, phenol resin, and silicon resin. Although examples of the grip part 212 other than the Q-shaped elastic member are not illustrated, the grip part 212 may be a clip which is openable and closable at a fulcrum. Instead of the Q shape, the grip part 212 may be semicircular in shape.

In addition to the above, wing parts 212a are provided at the disconnected part of the ring in the grip part 212. These wing parts 212a, for example, make contact with the puncture position 30 so as to stabilize the posture of the electrode connector 21 with respect to the puncture position 30 (e.g., prevent the electrode connector 21 from tilting leftward or rightward). The width (size) of each wing part 212a may be suitably determined in accordance with an installation location. When the grip part 212 is detached from the puncture appliance 31, the grip part 212 is easily detached because this part is an elastic member. Alternatively, the grip part 212 may be detached from the puncture appliance 31 by gripping and widening the wing parts 212a.

As such, when the puncture appliance 31 puncturing the puncture position 30 deviates and an abnormal installation state such a as the removal of the puncture appliance 31 occurs, the electrode connector 21 attached to the puncture appliance 31 by the grip part 212 is disconnected from the electrode members 15 so that the liquid detecting device 10 can detect the disconnected state. As a result, the liquid detecting device 10 is able to detect the occurrence of the abnormal installation state of the puncture appliance 31 at least by detecting the disconnected state.

### (Electric Structure of Liquid Detecting Device)

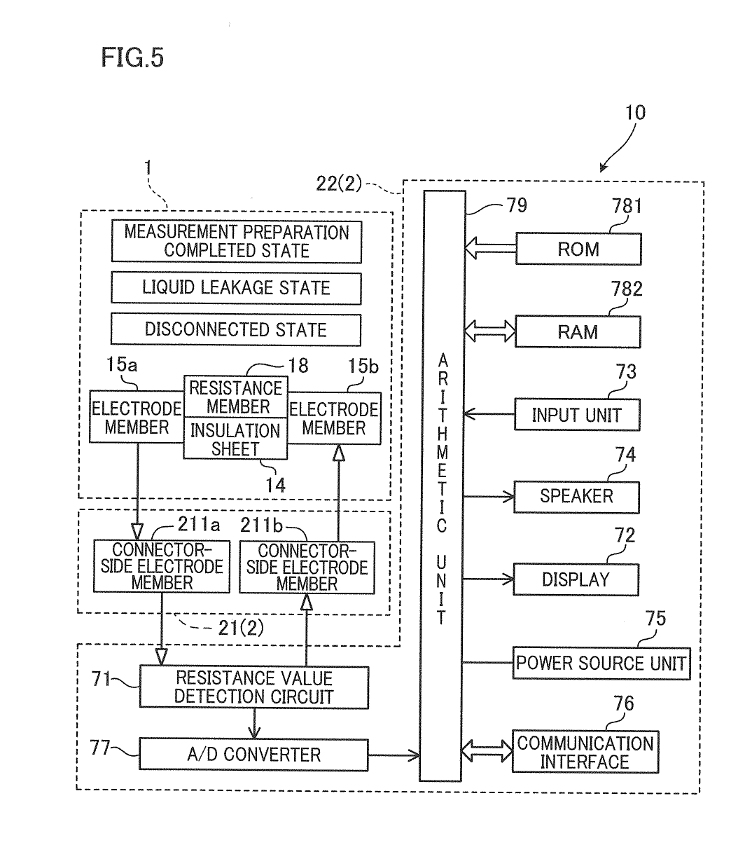

Now, the electric structure of the liquid detecting device 10 will be described with reference to FIG. 5. In the liquid detection sensor 1, a circuit in which the electrode members 15a and 15b are connected with each other by the resistance member 18 is formed on the insulation sheet 14. For example, when the insulation sheet 14 exhibits conductivity on account of the presence of liquid, a circuit in which the electrode members 15a and 15b are (electrically) connected with each other by the insulation sheet 14 is formed. Note that the resistance member 18 and the insulation sheet 14 may be independently connected with the electrode members 15a and 15b.

The main body 22 of the measurement device 2 includes an arithmetic unit 79, a resistance value detection circuit 71, an A/D converter 77, an input unit 73, a speaker 74, a display 72, a power source unit 75, a communication interface 76, a ROM 781, and a RAM 782.

The resistance value detection circuit 71 is connected with the electrode members 15a and 15b of the liquid detection sensor 1 via the connector-side electrode members 211a and 211b of the electrode connector 21. Based on electric power from the power source unit 75, the resistance value detection circuit 71 applies a predetermined voltage between the electrode members 15a and 15b, and measures a current output from the electrode members 15 by using an unillustrated ammeter. Furthermore, based on this current and the applied voltage, the resistance value detection circuit 71 calculates a value of resistance between the electrode members 15a and 15b. In other words, in the resistance value detection circuit 71, the value of resistance of the resistance member 18 between the electrode members 15a and 15b is calculated in the measurement preparation completed state. Furthermore, in the liquid leakage state, a value of resistance of the insulation sheet 14 exhibiting conductivity is detected. Furthermore, in the disconnected state, because the electrical connection between the electrode member 15a and the electrode connector 211a and/or between the electrode member 15b and the connector-side electrode member 211b is released, no current flows and hence the value of resistance is infinite. The resistance value detection circuit 71 then outputs the calculated value of resistance to the arithmetic unit 79 via the A/D converter 77.

With the electric power supply from the power source unit 75, the arithmetic unit 79 executes programs and control the operation of each of actuators. To be more specific, the arithmetic unit 79 executes a later-descried liquid leakage detection program to determine the state of the liquid detection sensor 1 based on the value of resistance from the resistance value detection circuit 71. Programs such as the liquid leakage detection program are stored in a storage such as the ROM 781 and the RAM 782. Furthermore, in accordance with the determined state of the liquid detection sensor 1, the arithmetic unit 79 notifies the state of the liquid detection sensor 1 by means of the speaker 74 or the display which is, for example, a liquid crystal display. Furthermore, by means of the input unit 73 such as a switch, a keyboard, and a mouse, it is possible to set the start and the end of detection, a threshold for the determination of the state of the liquid detection sensor 1, etc. Such setting values are stored in the RAM 782.

In addition to the above, the arithmetic unit 79 is able to output, to the outside, an identification information signal based on the identification information which indicates the state of the liquid detection sensor 1, via the communication interface 76. To put it differently, the communication interface 76 functions as a terminal-side communication unit which transmits at least the identification information. To be more specific, the arithmetic unit 79 transmits, via the communication interface 76, unique ID (Identification) information together with the identification information. The unique ID information is information for specifying each measurement device 2. In the present embodiment, the measurement device 2 transmits the identification information signal to the monitoring device 11 in the liquid detecting system 20. For example, when liquid leakage from an installation target is detected, a signal can be output to a system such as the monitoring device 11. This makes it possible to, for example, send a alarm to a remote place or perform automatic response to the liquid leakage (e.g., automatically stopping a system related to the installation target). As such, it is possible to specify which measurement device 2 is the source of data transmission, based on the ID information. Because the liquid detection sensor 1 in an abnormal state can be specified, it is possible to remotely monitor the liquid detecting devices 10 when the ID information is preliminarily associated with each installation location. In the present embodiment, as described above, the identification information signal is output to the outside by wireless communication. Alternatively, the measurement device 2 may be further provided with an external contact output for wired communication, as the terminal-side communication unit. This allows the terminal-side communication unit to support both wired communications and wireless communications.

### (Monitoring Device)

As shown in FIG. 2, the monitoring device 11 is configured to monitor the liquid detecting devices 10. To be more specific, the monitoring device 11 includes: a monitoring communication part 111 which is connected with the communication interface 76, as terminal-side communication unit of the liquid detecting device 10, to perform data communications with the communication interface 76; a monitoring storage part 112 which is configured to store the ID information of each liquid detecting device 10 in association with installation location information; a display 113 which is configured to demonstrate the identification information and the installation location information; and a display processor 114 which is configured to cause the display 113 to demonstrate the identification information input by monitoring communication part 111 and the installation location information which is associated with the ID information input together with the identification information.

The monitoring device 11 is a computer and includes a CPU (Central Processing Unit), a ROM storing programs executed by the CPU and data used by the programs, and a RAM temporarily storing data when a program is executed. The RAM functions as the monitoring storage part 112. Furthermore, the monitoring device 11 includes input devices such as a switch, keyboard, and a mouse, and is provided with a liquid crystal display as the display 113. These components included in the monitoring device 11 are constructed by the cooperation of the aforesaid hardware and the software and data in the ROM. The monitoring device 11 may not be a single computer, and the functions of the aforesaid components may be distributed to a plurality of computers, mobile devices, PDAs, etc.

### (Monitoring Device: Installation Information Table)

The following will describe an installation information table stored in the monitoring storage part 112.

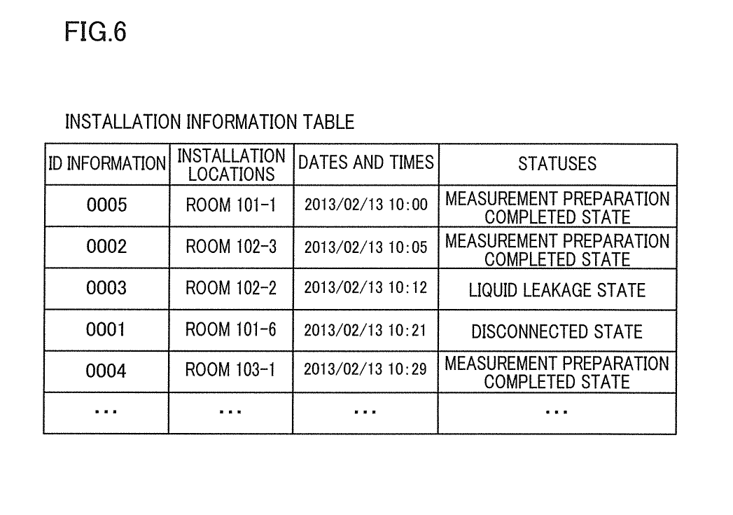

As shown in FIG. 6, the installation information table includes an ID information column, an installation location column, a start date and time column, and a status column. The ID information column lists ID information for identifying each measurement device 2. The installation location column lists information indicating the installation location of each measurement device 2 (liquid detecting device 10). When each measurement device 2 is associated with an installation location in a fixed manner, the ID information and the installation location information may be stored in association with the monitoring device 11 in advance. In the meanwhile, when the measurement device 2 is used in plural installation locations, an installation location may be input to the measurement device 2 at the start of the measurement in order to include the installation location information in the identification information signal. Alternatively, the installation location may be input to the monitoring device 11 at the start of the measurement. The start date and time column lists a date and time of the start of the measurement. The date and time may be input when the identification information signal is received for the first time, or may be input to the monitoring device 11 at the start of the measurement. The status column lists the state (the liquid leakage state, the disconnected state, or the measurement preparation completed state) of the liquid detection sensor 1 measured by the measurement device 2. In the monitoring device 11, when an unused measurement device 2 is managed, information indicating the unused state may be input to the status column. In such a case, an input indicating that the measurement for a used measurement device 2 has been completed may be made to the measurement device 2 or the monitoring device 11.

### (Application Example of Liquid Detection Sensor)

Plurality of Sheet-shaped liquid detection sensors 1 produced as described above are stacked together. These liquid detection sensors 1 are stored in a storage such as a pocket of a worker, a tool container, etc. In other words, the liquid detection sensors 1 can be stored to be carried by a worker in the same manner as adhesive plasters with gauze.

As shown in FIG. 1, in case where the installation subject of equipment and location that require liquid leakage check exists, peeling the peelable sheet 13 if the liquid detection sensor 1 has the peelable sheet 13, or directly pasting it onto the puncture position 30 if the liquid detection sensor 1 does not have the peelable sheet 13.

Subsequently, the electrode members 15a and 15b which are exposed to the outside in the liquid detection sensor 1 are lifted up together with the insulation sheet 14, and are then sandwiched between the sandwiching parts 214a and 215a of the electrode connector 21 of the measurement device 2. With this, the connector-side electrode members 211a and 211b of the sandwiching part 214a of the electrode connector 21 are electrically connected with the electrode members 15a and 15b, respectively. The electrode connector 21 is fixable, by the grip part 212, to the puncture appliance 31 which is the installation target. Furthermore, the grip part 212 has the wing parts 212a, and this makes it possible to stably install the electrode connector 21 to the puncture position 30.

As such, the liquid detection sensor 1, which includes the insulation sheet 14 which exhibits conductivity in the presence of liquid, the electrode members 15 which are provided on one surface of the insulation sheet in a contacting manner and are electrically isolated from each other, and the resistance member 18 which is connected to join the electrode members together, is set on the liquid leakage detection target. Then the electrode members 15 of the liquid detection sensor 1 are detachably connected via the electrode connector 21.

When liquid leakage occurs in the installation target, the insulation sheet 14 exhibits conductivity on account of the permeation of the liquid. With this, the electrode members 15a and 15b which had been electrically connected with each other only by the resistance member 18 become electrically connected with each other also by the insulation sheet 14. Because the value of resistance of the insulation sheet 14 exhibiting conductivity is lower than that of the resistance member 18, the electric resistance indicated by the measurement device 2 also becomes lower. In this way, the liquid leakage state is detected.

When the connection between the resistance member 18 and the electrode members 15 or the connection between the electrode members 15 and the measurement device 2 is released for some reason, the electric resistance measured by the measurement device 2 becomes higher than the value of resistance of the resistance member 18. In this way, the disconnected state of the liquid detection sensor 1 is detected.

When such an abnormality due to liquid leakage occurs, the liquid detection sensor 1 having detected the liquid is peeled off from the installation target by the worker, and the measurement is terminated. When the measurement is performed again, the liquid detection sensor 1 is replaced with an unused one to reinstate the measurement preparation completed state. As such, the liquid detection sensor 1 can be used in a disposable manner as in the case of adhesive plasters with gauze. The used liquid detection sensor 1 may be reused after the permeated liquid is dried. In the disconnected state, the electrode members 15 of the liquid detection sensor 1 are connected via the electrode connector 21 by the worker, so that the liquid detection sensor 1 is set to the measurement preparation completed state again.

### (Operations of Liquid Detecting Device)

Next, the liquid leakage detection program which is run by the arithmetic unit 79 of the measurement device 2 in the liquid detecting device 10 will be described. Note that, in the present embodiment, a value of resistance between the resistance member 18 and the electrode members 15a and 15b will be referred to as a value of sensor resistance.

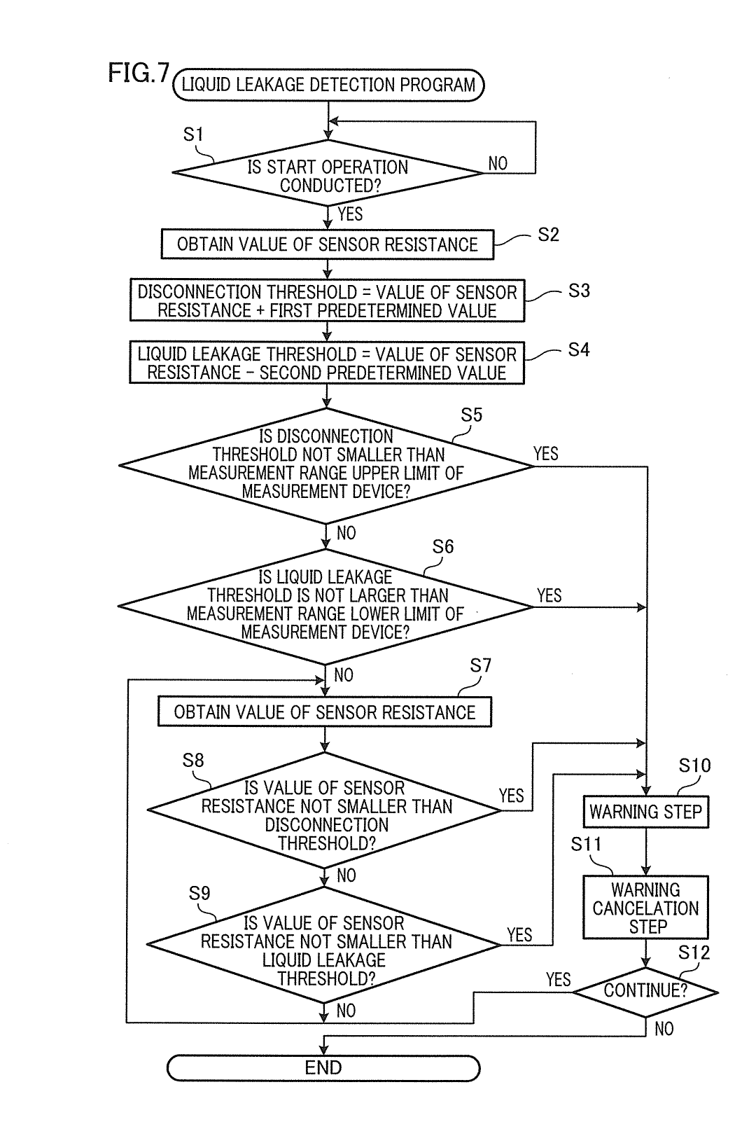

As shown in FIG. 7, First, whether a start operation has been conducted is determined (S1). To be more specific, the start operation is instructed from the outside by means of the input unit 73, and whether a signal indicating the operation has been transmitted to the arithmetic unit 79 is determined. When the start operation is not conducted (S1: NO), the step S1 is executed again. In other words, it is a standby state that wait for start operation.

In the meanwhile, when the start operation is conducted (S1: YES), a value of sensor resistance is obtained (S2). Thereafter, a disconnection threshold is determined (S3). To be more specific, the disconnection threshold is a threshold for identifying the disconnected state in which the connection between the electrode members 15a and 15b and the measurement device 2 (resistance value detection part 23) is released or poor. As described above, the disconnected state is a state in which the electrode connector 21 is disconnected from the electrode members 15a and 15b. When the electrode connector 21 is fixed to the puncture appliance 31, the disconnected state is a state in which the puncture appliance 31 is separated from the puncture position 30 (e.g., an indwelling needle, a winged needle, etc. falls off in the case of drip infusion). The disconnection threshold may be set to a value which the electrode members 15a and 15b are completely electrically isolated from each other (i.e., the value of resistance is infinite). In the present embodiment, the disconnection threshold is calculated by adding a first predetermined value to the value of sensor resistance when the liquid detection sensor 1 is installed to the installation target. The first predetermined value is suitably determined in advance in accordance with the environment of the installation target.

Then a liquid leakage threshold is determined (S4). To be more specific, the liquid leakage threshold is a threshold for identifying the liquid leakage state in which the insulation sheet 14 exhibits conductivity owing to liquid leakage and the electrode members 15a and 15b are electrically connected with each other by the insulation sheet 14. In the liquid detection sensor 1, because the value of resistance of the resistance member 18 is configured to be higher than the value of resistance of the insulation sheet 14 exhibiting conductivity, the liquid leakage threshold is set at a value of resistance between the value of resistance of the insulation sheet 14 exhibiting conductivity and the value of resistance of the resistance member 18. In the present embodiment, the liquid leakage threshold is calculated by subtracting a second predetermined value from the value of sensor resistance when the liquid detection sensor 1 is installed to the installation target. The second predetermined value is suitably determined in advance in accordance with the physical properties of the insulation sheet 14 and the liquid which is the target of leakage detection.

The liquid leakage threshold may be selected from a plurality of options. For example, the second predetermined value may be selectable from options, in the measurement device 2. To be more specific, the liquid detecting device 10 above was constructed and the relationship between the liquid leakage state of the liquid detection sensor 1 and the value of resistance of the liquid detection sensor 1 was measured. As a result, the value of resistance ranging between 5 kΩ and 6 kΩ was detected when a physiological salt solution for an amount of 0.05 ml was added to the insulation sheet 14, and the value of resistance ranging between 2 kΩ and 3 kΩ was detected when a physiological salt solution for an amount of 0.10 ml was added to the insulation sheet 14. As such, in the liquid detecting device 10, the liquid leakage threshold may be selectable from 6 kΩ and 3 kΩ. To put it differently, 6 kΩ is selectable as the liquid leakage threshold when a small amount of the physiological salt solution is to be detected, whereas 3 kΩ is selectable as the liquid leakage threshold when a large amount of the physiological salt solution is to be detected. In addition to the above, the disconnection threshold may be selectable from plural options. When the detection target is blood or the like, the blood is taken as a sample. t Then, a threshold for liquid leakage detection is suitably set based on a measurement result of the sample.

As described above, because a threshold is individually set for each liquid detection sensor 1, variations in the values of resistance are corrected even if the liquid detection sensors 1 have different values of resistance.

Thereafter, whether the liquid detection sensor 1 is in the liquid leakage state is determined. To be more specific, whether the determined disconnection threshold is not lower than a measurement range upper limit of the measurement device (S5). Note that, when it is allowed to set the disconnection threshold to infinite, this step may not be executed. When the determined disconnection threshold is lower than the measurement range upper limit of the measurement device (S5: NO), whether the determined liquid leakage threshold is not higher than a measurement range lower limit of the measurement device (S6). When the determined liquid leakage threshold is higher than the measurement range lower limit of the measurement device (S6: NO), a detection operation starts.

When the detection operation starts, a value of sensor resistance is obtained (S7). Then whether the obtained value of sensor resistance is not lower than the disconnection threshold is determined (S8). When the value of sensor resistance is lower than disconnection threshold (S8: NO), whether the obtained value of sensor resistance is not lower than the liquid leakage threshold is determined (S9). When the value of sensor resistance is lower than the liquid leakage threshold (S9: NO), the process goes back to the step S7 and the detection operation is continued.

In the meanwhile, when an abnormality is identified in each of the abnormality detection steps S5, S6, S8, and S9, (i.e., when the disconnection threshold is not lower than the measurement range upper limit of the measurement device (S5: YES), when the liquid leakage threshold is not higher than the measurement range lower limit of the measurement device (S6: YES), when the value of sensor resistance is not lower than the disconnection threshold (S8: YES), or the value of sensor resistance is not lower than the liquid leakage threshold (S9: YES)), the next step is executed.

In other words, when an abnormality is identified in each of the abnormality detection steps S5, S6, S8, and S9, a warning step is executed (S10). To be more specific, the speaker 74 is controlled to output alarm sound, and image display indicating the identification information is performed on the display 72. Furthermore, the identification information signal is transmitted to the monitoring device 11.

When receiving the identification information signal, the monitoring device 11 causes the display 113 to perform image display indicating the identification information. To be more specific, the monitoring device 11 obtains ID information included in the identification information signal. Thereafter, the monitoring device 11 searches the installation information table for location information corresponding to the obtained ID information, and updates the status column of the installation information table. The monitoring device 11 then causes the display 113 to display the ID information indicating the measurement device 2, the location information of the location where the measurement device 2 is provided, and the state of the liquid detection sensor 1 measured by the measurement device 2. As such, it is possible to monitor the liquid detecting device 10 even if the liquid detecting device 10 is remote from the monitoring device 11. Note that, when the monitoring device 11 causes the display 113 to display the state of the liquid detection sensor 1, sound may be output from a speaker or like.

Furthermore, in an unillustrated case where the measurement device 2 is connected with a dialyzer to be able to perform data communications therewith, the identification information signal may be transmitted to the dialyzer in S10. The dialyzer is preferably configured such that, when the identification information signal indicating the disconnected state or the liquid leakage state is received, a pump of the dialyzer by which blood is circulated is stopped. This makes it possible to quickly restrain the liquid leakage from, for example, the puncture position where the liquid detection sensor 1 is installed and the liquid leakage due to improper installation of a puncture appliance such as falloff of the needle.

After S10, a warning cancellation step is executed (S11). The warning cancellation step is a step in which the warning step above is terminated when an instruction to cancel the warning is input to the input unit 73 by an administrator or the like of the liquid detection sensor 1. Furthermore, a warning cancellation signal is transmitted to the monitoring device 11.

Upon receiving the warning cancellation signal, the monitoring device 11 may terminate the display of the state of the liquid detection sensor 1. Alternatively, upon receiving the warning cancellation signal, the monitoring device 11 may display information indicating that the abnormality in the liquid detecting device 10 has been resolved.

After S11, whether to continue the detection operation is determined (S12). To put it differently, whether an input to the input unit 73 from an administrator or the like of the liquid detection sensor 1 indicates the continuation of the current detection operation is determined. In addition to this, the selection of one of the inputs above may be displayed on the display 72.

When the input indicates the continuation of the current detection operation (S12: YES), the process goes back to the step S7 and the detection operation is continued. When the input does not indicate the continuation of the current detection operation (S12: NO), the program is terminated.

As such, a resistance value detection step of detecting a value of resistance between the electrode members 15, a state determination step of discriminating between the liquid leakage state in which the insulation sheet 14 exhibits conductivity based on the value of resistance between the electrode members 15, the disconnected state in which the electrode members 15 are disconnected from the resistance value detection part 23, and the measurement preparation completed state which is different from these state, and an information output step of outputting the identification information corresponding to the state identified by the state identification part 24 are executed.

In addition to the above, the liquid leakage detection program may have a timer function. That is to say, a finish time may be registered at the start of the liquid leakage detection program, and an interruption to finish the liquid leakage detection program may be performed when the finish time comes. Alternatively, time counting may start and an execution time may be registered at the start of the liquid leakage detection program, and an interruption to finish the liquid leakage detection program may be performed when the counted time reaches the registered execution time. Furthermore, a registered finish time may be sent to the monitoring device 11, and the monitoring device 11 may send notification at the finish time.

In addition to the above, each time a predetermined time elapses (e.g., after the result of the determination in S9 is NO), the liquid leakage detection program may perform an output, to the monitoring device 11, of a identification information signal which indicates that the measurement preparation completed state has been set. This allows the monitoring device 11 to detect that the measurement device 2 itself malfunctions, when the regularly-sent identification information signal is not received.

Furthermore, when the result of the determination in S12 is NO, the liquid leakage detection program may perform an output, to the monitoring device 11, of a signal indicating that the measurement has been finished.

The electrode connector 21 may be provided with an LED which indicates, by continuous light emission or blinking, that the state of the liquid detection sensor is the measurement preparation completed state, the liquid leakage state, or the disconnected state. To be more specific, the LED blinks when the electrode connector 21 is not attached to or not properly attached to the electrode members 15a and 15b, continuously emits light when the electrode connector 21 is properly attached to the electrode members 15a and 15b, or blinks in the liquid leakage state or in the disconnected state and at the same time warning sound is output from the main body 22 (detector) side. In this case, the measurement device 2 includes, for example, a controller which receives the identification information signal output in the step S10 and controls the LED to react as above based on the received signal. With this configuration, the state of the LED is changed from blinking to continuous light emission when the electrode connector 21 is attached to the electrode members 15a and 15b. It is therefore possible to visually and immediately recognize whether the electrode connector 21 is properly attached at the moment of installing. This prevents the occurrence of a failure in the installation. Furthermore, as compared to a case where the state of the installation is checked by an LED on the main body 22 (detector) side, it is unnecessary to significantly move the line of sight from the currently-operated electrode connector 21, and hence the state of the attachment is quickly recognizable and the usability is improved.

In addition to the above, a display such as an LED may be provided on a holder of the electrode connector to indicate whether the connection between the electrode members of the liquid detection sheet and the electrode members of the electrode connector is in the measurement preparation completed state (normal state) or the disconnected state (detached state: a state in which the connector-side electrode members are disconnected from the electrode members of the liquid detection sheet). The normal state and the detached state may be indicated by (continuous light emission and blinking of) the LED provided on the holder.

In addition to the above, while in the present embodiment the grip part is Q-shaped in cross section, the grip part may be differently shaped as long as it has a ring shape disconnected at one part in cross section. For example, the grip part may be C-shaped, U-shaped, or Q-shaped. The electrode connector and the grip part may be integrally molded. Alternatively, although not illustrated, the electrode connector and the grip part may be joined with each other in a ball joint manner to allow the grip part to be rotatable, or a clip-type so that the grip part is able to open and close by a fulcrum.

The detailed description of the present invention provided hereinabove mainly focused on characteristics thereof for the purpose of easier understanding; however, the scope of the present invention shall be construed as broadly as possible, encompassing various forms of other possible embodiments, and therefore the present invention shall not be limited to the above description.

Further, the terms and phraseology used in the present specification are adopted solely to provide specific illustration of the present invention, and in no case should the scope of the present invention be limited by such terms and phraseology. Further, it will be obvious to those skilled in the art that the other configurations, systems, methods and the like are possible, within the spirit of the invention described in the present specification. The description of claims therefore shall encompass configurations equivalent to the present invention, unless otherwise such configurations are regarded as to depart from the spirit and scope of the present invention. To fully understand the object and effects of the present invention, it is strongly encouraged to sufficiently refer to disclosures of documents already made available.

### REFERENCE SIGNS LIST

1 LIQUID DETECTION SENSOR
2 MEASUREMENT DEVICE
2a CORD
10 LIQUID DETECTING DEVICE
11 MONITORING DEVICE
13 PEELABLE SHEET
14 INSULATION SHEET
15 ELECTRODE MEMBER
16 ADHESIVE MEMBER
18 RESISTANCE MEMBER
19 ADHESIVE LAYER
20 liquid detecting system
21 ELECTRODE CONNECTOR
22 MAIN BODY
23 RESISTANCE VALUE DETECTION PART
24 STATE IDENTIFICATION PART
25 INFORMATION OUTPUT PART
30 PUNCTURE POSITION
31 PUNCTURE APPLIANCE
61 ADHESIVE
62 ADHESIVE FILM
71 RESISTANCE VALUE DETECTION CIRCUIT
72 DISPLAY
73 INPUT UNIT
74 SPEAKER
75 POWER SOURCE UNIT
76 COMMUNICATION INTERFACE
77 A/D CONVERTER
79 ARITHMETIC UNIT
111 MONITORING COMMUNICATION PART
112 MONITORING STORAGE PART
113 DISPLAY
114 DISPLAY PROCESSOR
151 CONDUCTIVE ADHESIVE LAYER
152 METAL LAYER
211 CONNECTOR-SIDE ELECTRODE MEMBER
211a CONNECTOR-SIDE ELECTRODE MEMBER
211b CONNECTOR-SIDE ELECTRODE MEMBER
212 GRIP PART
212a WING PART
213 FULCRUM
214 HOLDER
214a SANDWICHING PART
215 HOLDER
215a SANDWICHING PART
781 ROM
782 RAM

1. A liquid detecting device comprising:
a liquid detection sensor including an insulation sheet which exhibits conductivity in the presence of liquid, a plurality of electrode members which are provided on one surface of the insulation sheet in a contacting manner and are electrically isolated from each other, and a resistance member connected to join the electrode members together;
a resistance value detection part which is detachably connected to the electrode members via a signal line and is configured to detect a value of resistance between the electrode members; a state identification part which identifies, based on the value of resistance between the electrode members, a liquid leakage state in which the insulation sheet exhibits conductivity, a disconnected state in which the electrode members are disconnected from the resistance value detection part, and a measurement preparation completed state; and
an information output part which is configured to output identification information which corresponds to each state identified by the state identification part.

2. The liquid detecting device according to claim 1, wherein, in the liquid detection sensor, the resistance member has a value of resistance which is higher than a value of resistance when the insulation sheet exhibits conductivity and lower than a value of resistance when the electrode members are disconnected from the resistance value detection part.

3. The liquid detecting device according to claim 2, wherein, the resistance value detection part includes an electrode connector which includes a sandwiching part which is able to sandwich the liquid detection sensor and a connector-side electrode member which is provided in the sandwiching part, is in contact with the electrode members when the liquid detection sensor is sandwiched, and is electrically connected with the signal line.

4. The liquid detecting device according to claim 3, wherein,
the liquid detection sensor is installed to a puncture position which is punctured by a puncture appliance, and
the electrode connector includes a grip part which is attachable to the puncture appliance.

5. The liquid detecting device according to claim 4, wherein, the information output part includes an output unit which is configured to output the identification information by sound and/or light.

6. The liquid detecting device according to claim 5, wherein, the information output part includes a terminal-side communication unit which is configured to transmit at least the identification information.

7. The liquid detecting device according to claim 6, wherein, the terminal-side communication unit is configured to transmit unique ID information together with the identification information.

8. A liquid detecting system comprising:
the liquid detecting device of claim 7; and
a monitoring device which is configured to monitor the liquid detecting device,
the monitoring device including:
a monitoring communication part which is connected with the terminal-side communication unit of the liquid detecting device to be able to perform data communications with the terminal-side communication part;
a monitoring storage part which is configured to store the ID information of the liquid detecting device in association with installation location information;
a display which is configured to display the identification information and the installation location information; and
a display processor which is configured to cause the display to display identification information which is input through the monitoring communication part and the installation location information which corresponds to the ID information input together with the identification information.

9. A liquid detecting method comprising:
a step of setting, to a liquid leakage detection target, a liquid detection sensor including an insulation sheet which exhibits conductivity in the presence of liquid, a plurality of electrode members which are provided on one surface of the insulation sheet in a contacting manner and are electrically isolated from each other, and a resistance member connected to join the electrode members together;
a resistance value detection step of establishing detachable connection with the electrode member via a signal line and detecting a value of resistance between the electrode members;
a state identification step of, based on the value of resistance between the electrode members, identifying a liquid leakage state in which the insulation sheet exhibits conductivity, a disconnected state in which the electrode members are disconnected from the resistance value detection part, and a measurement preparation completed state; and
an information output step of outputting identification information which corresponds to each state identified by the state identification part.

10. An electrode connector configured to detect a value of resistance between electrode members and detachably connected to the electrode members via a signal line, wherein,
the electrode members are provided in a contacting manner on one surface of an insulation sheet which is installed to a puncture position punctured by a puncture appliance and exhibit conductivity in the presence of liquid, and are electrically isolated from each other,
the electrode connector comprising a grip part which is attachable to the puncture appliance.
